(19) United States
(12) Patent Application Publication
(10) Pub. No.: US 2004/0249264 A1
(43) Pub. Date: Dec. 9, 2004

(54) Medical triggering device

(76) Inventors: Ivan S. Salgo, Andover, MA (US); John W. Goodnow, Arlington, MA (US); Joseph Savino, Moorestown, NJ (US)

Correspondence Address: PHILIPS INTELLECTUAL PROPERTY & STANDARDS, P.O. BOX 3001, BRIARCLIFF MANOR, NY, 10510, US

(21) Appl. No.: 09/919,237

(22) Filed: Jul. 31, 2001

(51) Int. Cl. A61B 5/05; A61B 5/08; A61B 8/00

(52) U.S. Cl. 600/413; 600/529; 600/428; 600/437

(57) ABSTRACT

A trigger generator for supplying a trigger signal to a medical device. The generator includes a respiratory signal device associated with the subject that generates a respiratory signal representing the flow of gas into and out of the subject's lungs during the subject's breathing cycle. A trigger generator integrates the respiratory signal and generates a trigger signal when the integrated respiratory signal has a value representing a selected point in the subject's breathing cycle.

### BACKGROUND OF THE INVENTION

The acquisition of physiologic data, such as intra cardiac pressures, and images, such as radiological, ultrasound, computed tomographic and magnetic resonance images, for medical purposes is a well known and commonly practiced process. Such medical data and image acquisition methods (hereinafter referred to simply as “data acquisition”), however, present certain recurring and persistent difficulties in that the normal and necessary operations of the human body interfere with the data acquisition by introducing motion, noise and other extraneous signals into the data to be acquired. The most common source of such interference is the respiratory process which causes changes in the position and orientation of organs, including the heart, and changes in gas and fluid pressures and compositions in the body as the lungs fill and empty.

The acquisition of physiologic data and images benefits from real time information regarding current lung volume so that a series of data acquisitions may be performed at the same lung volume in the hope that the organ of interest, such as the heart, is in the same position and orientation for each acquisition. Generally, it is preferable that the data be acquired when the lungs, and thus the organ of interest, are not in motion due to the filling or emptying of the lungs.

Interference from the respiratory operations of the body has generally been eliminated in the prior art either by suspending the respiratory operation or by attempting to sense the subject's current lung volume. Suspension of the respiratory function comprises, for example, having the subject “hold their breath” or briefly turning off the respiratory process when a subject's breathing is externally controlled. However, suspension may induce an artificial state that may affect the validity of the physiologic variable or data of interest. Suspension might also be potentially hazardous to the subject. Further, it is not unusual that a subject is physically incapable of holding their breath (at least for the necessary time).

It is known to estimate lung volume by sensing either the flow of respiratory gases or a physiologic variable that is affected by the change in volume of the lungs. Examples of commonly used techniques include: spectrophotometric measurement of CO₂ by a capnogram to detect the difference between fresh gas as a subject inhales and the CO₂ laden gas that the subject exhales; the use of an EKG to measure changes in transthoracic impedance as the subject inhales and exhales; and spirometry to measure gas flow velocity as the subject inhales and exhales.

The method used to estimate lung volume in any instance will depend, of course, upon the particular circumstances. For example, asking a subject to hold their breath requires no complex or expensive equipment but using an EKG to sense changes in transthoracic impedance is generally more reliable and does not depend upon the conscious cooperation and capabilities of the subject who may, for example, be capable of only limited cooperation. However, the measurement of transthoracic impedance by EKG is vulnerable to noise incurred by subject movement and is unusable during thoracic or cardiac surgery when the chest wall is opened and fixed as there is little or no change in transthoracic impedance during breathing, even though there is variation of lung volume.

Consequently, a capnogram or similar device, such as a spirometer or spectrophotometric device, is frequently preferred over breath holding or an EKG as being the most reliable solution. For example, as noted, thoracic impedance is unusable during open heart surgery practically requiring the use of a capnogram or similar device. Capnograms and similar devices do not depend upon the conscious cooperation and capabilities of the subject and, as such, provide a highly stable waveform representing breathing activity that is not as susceptible to noise or affected by procedures involving thoracic wall entry as an EKG.

The recurring problem with a capnogram, and virtually all similar methods and devices for estimating lung volume, is that they measure, detect or otherwise respond to some factor (typically CO₂) in the flow of gas into and out of the lungs and thus to the change, or rate of change, in lung volume, rather than being directly dependent upon lung volume. Known gas flow dependent devices provide an electrical output that approximates a square or rectangular waveform. Unfortunately, the maximum and minimum values of these signal do not represent the points of maximum or minimum volume of gas in the lungs, but instead represent the periods during which gas is flowing into or out of the lungs. The periods that are usually of most interest for the acquisition of physiologic data are the periods during which there is the maximum or minimum volume of gas in the lungs. These periods occur during the transition portion of such waveforms between the maximum and minimum values. The interval occupied by this transition is usually very brief compared to the overall period of the waveform and the rate of change of the signal is very rapid during this period, that is, the slope of the signal is usually very steep during this period. As a consequence, it is difficult to detect the signal values representing the points of maximum or minimum gas volume in the lungs, and to reliably trigger the data acquisition devices upon these values.

The present invention provides a solution to these and other problems of the prior art.

### BRIEF DESCRIPTION OF THE DRAWINGS

These and other objects and advantages of the invention will become apparent and more readily appreciated from the following description of the preferred embodiments, taken in conjunction with the accompanying drawings of which:

FIG. 1 is a diagram of a data acquisition system in accordance with a preferred embodiment of the present invention;

FIG. 2A shows an example of a respiratory signal output by a respiratory signal device;

FIG. 2B is a chart of lung volume in accordance with a preferred embodiment of the present invention;

FIG. 2C is a chart of the change in lung volume over change in time in accordance with a preferred embodiment of the present invention;

FIG. 2D shows an example of an integrated respiratory signal output by an integrator in accordance with a preferred embodiment of the present invention; and

FIG. 3 is a diagram of a trigger generator in accordance with a preferred embodiment of the present invention.

### DESCRIPTION OF THE PREFERRED EMBODIMENTS

Reference will now be made in detail to the present preferred embodiments of the present invention, examples of which are illustrated in the accompanying drawings, wherein like reference numerals refer to like elements throughout.

The detailed description which follows is partially presented in terms of routines and symbolic representations of operations of data bits within a memory, associated processors, and possibly networks, and network devices. These descriptions and representations are the means used by those skilled in the art effectively convey the substance of their work to others skilled in the art. A routine is here, and generally, conceived to be a self-consistent sequence of steps or actions leading to a desired result. Thus, the term “routine” is generally used to refer to a series of operations performed by hardware components including dedicated circuits or a processor, be it a central processing unit of a medical device, such as an ultrasound system, or a secondary processing unit of such an ultrasound system, and as such, encompasses such terms of art as “program,” “objects,” “functions,” “subroutines,” and “procedures.”

In general, the sequence of steps in the routines require physical manipulation of physical quantities. Usually, though not necessarily, these quantities take the form of signals, optical, electrical or magnetic, capable of being stored, transferred, combined, compared or otherwise manipulated. Those of ordinary skill in the art conveniently refer to these signals as “bits”, “values”, “elements”, “symbols”, “characters”, “images”, “terms”, “numbers”, or the like. It should be recognized that these and similar terms are to be associated with the appropriate physical quantities and are merely convenient labels applied to these quantities.

While the apparatus set forth in the present application may be specifically constructed for the required purpose, the methods recited herein may be implemented on a general purpose computer or other network device selectively activated or reconfigured by a routine stored in the computer and, if necessary, interface with the necessary external data acquisition equipment. In particular, various machines may be used with the methods of the present invention in accordance with the teachings herein, or it may prove more convenient to use dedicated hardware, such as ASICs, to perform the required method steps. One example of such dedicated hardware, using discrete components, will be discussed herein, but those of ordinary skill in the art will recognize the applicability of other devices, both specialized and general. Machines which may perform the functions of the present invention include those manufactured by such companies as AGILENT TECHNOLOGIES, PHILIPS MEDICAL SYSTEMS INTERNATIONAL, GE MEDICAL SYSTEMS, and SIEMENS MEDICAL SYSTEMS, as well as other manufacturers of medical equipment.

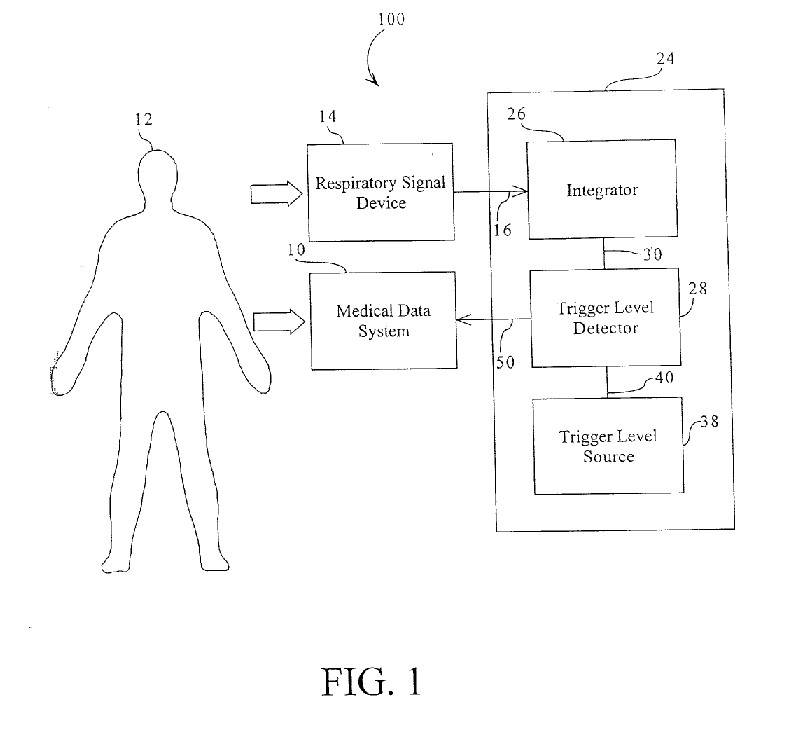

FIG. 1. is a diagrammatic representation of a medical data acquisition system 100 in accordance with a preferred embodiment of the present invention. Specifically, FIG. 1, is a logical representation of the medical data acquisition system 100 capable of performing a method for the acquisition of physiologic data that is simpler, more reliable and less costly than the apparatus and methods of the prior art. As will be described, the data acquisition system 100 facilitates the acquisition of physiologic data at selectable points in a subject's breathing cycle. Further, the present invention enables the selection of triggering point based on the volume of gas in a subject's lungs, rather than the rate of change of gases in the subject's lungs. In general, the medical data acquisition system 100 comprises a medical data system 10, a respiratory signal device 14 and a trigger generator 24.

The medical data system 10 acquires physiologic data, such as intra cardiac pressures or images (in the case of radiological, ultrasound, computed tomographic or magnetic systems), associated with a subject 12. The acquisition of such physiologic data may, in the manner usual for the particular medical data system 10, be triggered using a trigger signal (in either analog or digital form). The manner in which various types of medical data systems 10 are associated with the subject 12 and are triggered is well understood by practitioners of ordinary skill in the arts will not be discussed further herein to avoid obscuring the present invention.

The respiratory signal device 14 for measuring, detecting or otherwise responding to and indicating a factor in the flow of gas into and out of the subject 12's lungs, such as pressure, temperature or gas composition, is associated with the subject 12. The respiratory signal device generates a respiratory signal 200 representing the flow of gas into or out of the lungs of subject 12. The respiratory signal device 14 comprise, for example, a capnogram, spirometer or spectrophotometric device.

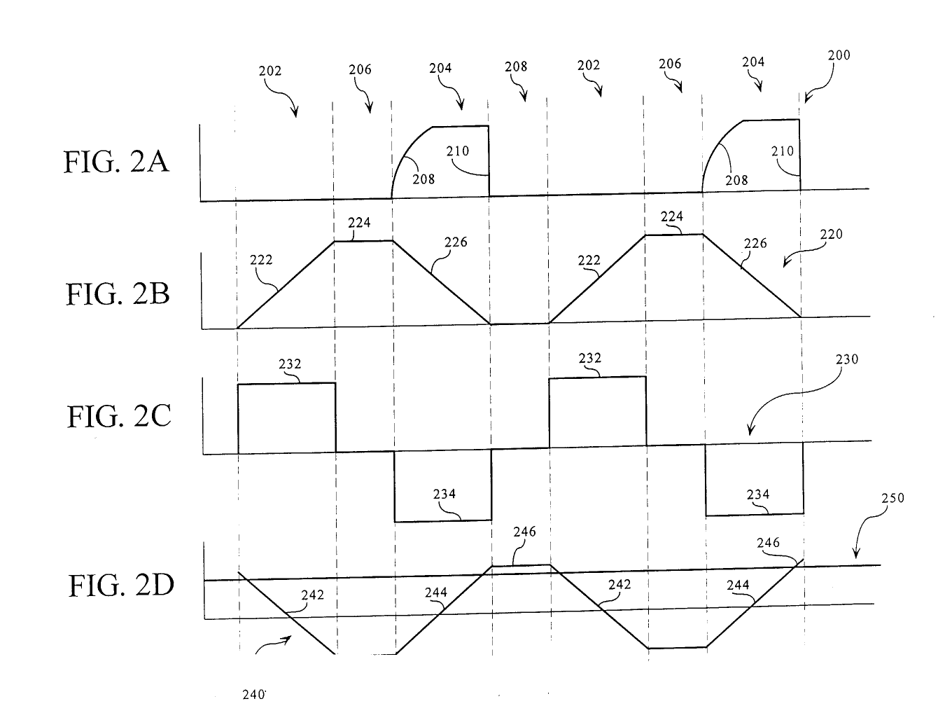

FIG. 2A shows an example of a respiratory signal 16, in this case a respiratory signal 200, output by a respiratory signal device 14 attached to a patient being ventilated. To be more specific the respiratory signal 200 approximates the output of a capnogram. Those of ordinary skill in the art will recognize that FIG. 2A is highly stylized for ease of explanation. Needless to say, the respiratory signal 200 may differ from one type of respiratory signal device 14 to another. The differences between the signals output by the various types of respiratory signal devices 14 are well understood by practitioners of ordinary skill in the arts and will not be discussed further herein to avoid obscuring the invention.

The respiratory signal 200 approximates a rectangular waveform. The waveform has a minimum value during an inhale period 202, a hold period 206 and a hold period 208. The waveform takes on a maximum value during an exhale period 204. The time encompassing the hold period 206 through the beginning of transition 208 represents the interval during which there is the maximum volume of gas in subject 12's lungs. The time extending from the end of the transition 210 through the hold period 208 represents the interval of minimum volume of gas in the lungs of the subject 12. The intervals occupied by transitions 208 and 210 are usually very brief compared to the overall period of respiratory signal 200 and the rate of change, e.g. the slope, of respiratory signal 200 during transitions 208 and 210 is usually very steep making triggering thereon problematic.

As discussed previously, the respiratory signal 200 does not directly indicate the points of maximum or minimum volume of gas in the lungs of the subject 12. FIG. 2B is a chart of lung volume 220 in accordance with a preferred embodiment of the present invention. The periods during which there is the maximum or minimum volume of gas in subject 12's lungs occurs when the respiratory signal 200 transitions between its maximum and minimum values. During the inhale period 202, the lungs of the patient 12 are being filled and the lung volume 220 enters a rising period 222. During the hold period 206, the lung volume 220 has constant period 224. During the exhale period 204, the lung volume 220 enters a descending period 226.

FIG. 2C is a chart of the change in lung volume over change in time in accordance with a preferred embodiment of the present invention. The graph has a maximum value during the period 232 during the inhalation period 202. Conversely, the graph 230 has a minimum value during the period 234 corresponding to the exhalation period 204. The graph 230 has a midline value during the periods of maximum and minimum volume.

Referring once again to FIG. 1, the respiratory signal 16, i.e the respiratory signal 200, is supplied to a trigger generator 24. In accordance with a preferred embodiment of the present invention, the trigger generator 24 comprises an integrator 26, a trigger level detector 28 and a trigger level source 38. The trigger generator 24 may be formed in software and/or hardware, depending on the desired implementation of the present invention. In general, the integrator 26, trigger level detector 28 and trigger level source 38 can be thought of a routines that can be implemented in software, to be executed by a processor, or in dedicated hardware.

The integrator 26 integrates, in any of a variety of known manners (including the use of both digital and analog methodologies), the respiratory signal 16, for example the respiratory signal 200, to generate an integrated respiratory signal 30. The integrator 26 may, as is known to those of ordinary skill in the art, be formed in circuitry or software to be executed by a processor (not shown). A discrete circuit has an advantage in that the signal from the respiratory signal device 24 need not be AMD converted prior to processing. In any event, the integrated respiratory signal 30 represents the time integrated form of respiratory signal 16.

FIG. 2D shows an example 240 of an integrated respiratory signal 30 output by an integrator 26 in accordance with a preferred embodiment of the present invention. While, the integrated respiratory signal 240 is shown in analog form for convenience, those of ordinary skill in the art will recognize that the integrated respiratory signal 30 can be equally represented in the digital domain with values stored in registers or general memory. As illustrated in FIG. 2B, Integrated respiratory signal 240 resembles the inverse of the lung volume of the patient 12. Significantly, the steepness of the slope of integrated respiratory signal 240 is significantly less than the slope of the respiratory signal 200 during transitions 208 and 210. The shape of the integrated respiratory signal 240 enables triggering based directly on the volume of gas in the lungs of the subject 12.

Referring once again to FIG. 1, the integrator 26 may have an integration period, or time constant, generally less than but close to the shorter of inhale period 202 or exhale period 204. This generates the maximum period for the transitions 242 and 244 while allowing the integrated respiratory signal 240 to reach and settle at its maximum and minimum values before the next transitions.

In certain configurations, the integrator 26 may operate as a low-pass filter, reducing the effect of noise in the respiratory signal 200. It will also be noted that certain respiratory signal devices 14 may add a constant DC offset voltage component to the respiratory signal 16. As a consequence, and as well understood by those of ordinary skill in the art, it may be preferable to remove the DC offset from the integration process. For example, a blocking capacitor may be inserted in the connection between the respiratory signal device 14 and the integrator 26, a compensating offset voltage could be applied, or in the case of a software solution, the offset can be compensated for as part of the integration routine.

The trigger detector 28 analyzes the integrated respiratory signal 30 and identifies the point in subject 12's respiratory cycle at which the medical data system 10 is to be triggered. The trigger detector 28 receives a trigger level signal 40 from the trigger level source 38. The trigger level signal 40 is variable based on input from a user of the data acquisition system 100. Typically, the trigger level corresponds to the amplitude of the integrated respiratory signal 30 at which user desires to trigger the operation of the medical data system 10. It is anticipated that most users will set the trigger level to correspond to the maximum or minimum volume of gas in the lungs of the subject. Thus, the trigger level detector 28, in its simplest form merely compares the value of the integrated respiratory signal 30 with the trigger level signal 40 and outputs a triggering signal 50, such as a pulse, when the respiratory signal 30 exceeds the trigger level 40. Of course more complicated criteria can be used as a predicate to generating a triggering pulse. For example, prior breathing cycles can be analyzed and a triggering point selected based on the analysis and the requirements of the medical data system 10.

FIG. 2D also shows an example 250 of a trigger level signal 40 output by the trigger level detector 28 in accordance with a preferred embodiment of the present invention. In this example, the trigger level detector 28 compares the integrated respiratory signal 240 to the trigger level signal 250 and generates a trigger signal 50 (for example, a pulse or increase/decrease in voltage or current) when the integrated respiratory signal 240 reaches the user selected level (MAX or MIN thresholds) represented by trigger level signal 40, in this example periods 246.

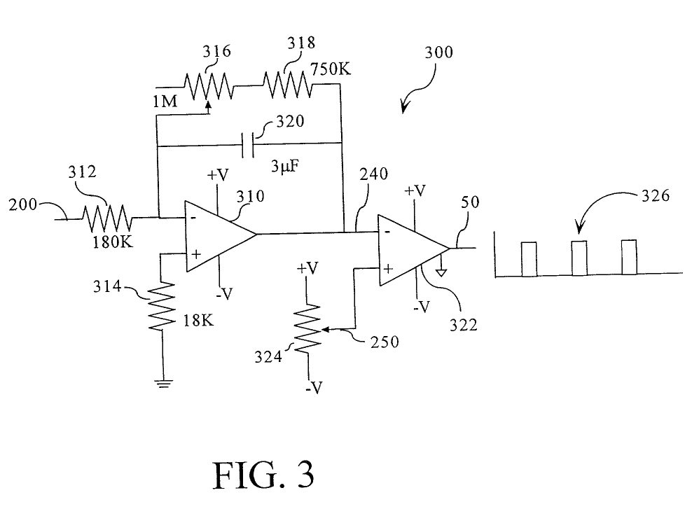

FIG. 3 illustrates a trigger generator 300 in accordance with a preferred embodiment of the present invention. The trigger generator 300 receives the respiratory signal 200 via a 180K Ohm resistor 312 which is in turn connected to an integrator op-amp 310, for example a TLD82. The negative input of the op-amp 310 is connected to ground through a 18K Ohm resistor 314. To avoid saturation of the op-amp 310 a 3 μF capacitor is connected in parallel therewith along with a 750K Ohm and a 1M Ohm variable resistor 316. The op-amp 310 output a signal corresponding to the integrated respiratory signal 240.

A comparator op-amp 322, for example a LM311, receives the integrated respiratory signal 240 and compares same to a value output by a potentiometer 324 that supplies the trigger level signal 40. The op-amp 50 output as pulse signal 326 corresponding to the period of minimal lung volume in the subject 12. The pulse signal 326 may serve as the trigger signal 50 (see. FIG. 1).

In the implementation shown in FIG. 3, it is assumed that the subject 12 has a ratio of inhale period 202 to exhale period 204 of 1:2, such as may occur when the subject 12 is under respirator controlled breathing and as opposed to the normal, uncontrolled ratio of 1:2 to 1:3. The op-amp 310 has an RC time constant of ⅙ Hz, corresponding generally to a respiratory rate of 10 breaths per minute, and a gain of unity. The trigger level signal 250 corresponds to the point in the integrated respiratory signal 200 representing the minimum volume of gas in the lungs of the subject 12.

Modifications are possible to adapt the circuit shown in FIG. 3 to different respiratory signal devices 14, to respiratory signals 16 having different duty cycles, to different medical data systems 10, and to different frequencies and different waveforms providing different voltage levels and waveforms for Trigger Signal 50. Such modification will be understood by those of ordinary skill in the arts in light of the present invention.

In the example shown in FIG. 3, the trigger signal 326 comprises a rectangular pulse. However, those of ordinary skill in the art will recognize that the trigger signal 50 may be of any usable value (in the case of a digital system) or shape (in the case of an analog system), including a rectangular or saw-tooth wave, suitable to trigger the operation of the medical data system 10 at the selected point in subject 12's respiratory cycle, as will be understood by those of ordinary skill in the arts.

Although a few preferred embodiments of the present invention have been shown and described, it would be appreciated by those skilled in the art that changes may be made in these embodiments without departing from the principles and spirit of the invention, the scope of which is defined in the claims and their equivalents. For example, while the trigger generator 24 is discussed as triggering the operation of a medical data system 10, it will be understood that the trigger generator 24 could be used to trigger any number of devices, such as a ventilator. Further, it is possible to trigger based on a differential of the respiratory signal, such as signal 230 in FIG. 2C. In this case triggering would be performed on every other mid-line period, between the period 234 and 232.

What is claimed is:

1. A trigger generator for supplying a trigger signal to a medical device, the trigger generator comprising:
a respiratory signal device associated with the subject that generates a respiratory signal representing the flow of gas into and out of the subject's lungs during the subject's breathing cycle; and
a trigger generator that integrates the respiratory signal and generates a trigger signal when the integrated respiratory signal has a value representing a selected point in the subject's breathing cycle.

2. The trigger generator of claim 1, wherein the trigger generator comprises:
an integrator that integrates the respiratory signal and generates a corresponding integrated respiratory signal; and
a trigger level detector that compares the integrated respiratory signal with a trigger level and generates the trigger signal when the integrated respiratory signal equals the trigger level.

3. The trigger generator of claim 2, wherein the trigger level detector further comprises:
a trigger level source for generating the trigger level representing the selected point in the subject's breathing cycle.

4. A method for triggering operation of a medical system at a selected point in a subject's breathing cycle, the method comprising the steps of:
receiving a respiratory signal representing the flow of gas into and out of the subject's lungs during the subject's breathing cycle;
integrating the respiratory signal to create an integrated respiratory signal; and
triggering the medical device when the integrated respiratory signal has a value corresponding to a selected point in the subject's breathing cycle.

5. The method of claim 4, wherein the steps of triggering the medical device comprises the steps of:
receiving the integrated respiratory signal;
receiving a trigger level representing a value corresponding to selected point in the subject's breathing cycle as described by an integrated signal;
comparing the integrated respiratory signal and the trigger level; and
generating the trigger signal when the integrated respiratory signal corresponds to the trigger value.

6. The method of claim 5, wherein the step of comparing the integrated respiratory signal and the trigger level signal comprises the step of setting a value to be represented by the trigger value.

7. A trigger generator for supplying a triggering to a medical device, the trigger generator comprising:
respiratory signal means associated with the subject for generating a respiratory signal representing the flow of gas into and out of the subject's lungs during the subject's breathing cycle; and
trigger generator means for integrating the respiratory signal and generating a trigger signal when the integrated respiratory signal has a value representing a selected point in the subjects breathing cycle.

8. A medical data acquisition system comprising:
a medical data system that acquires a set of data from a subject based on a trigger signal;
a respiratory signal device associated with the subject that generates a respiratory signal representing the flow of gas into and out of the subject's lungs during the subject's breathing cycle; and
a trigger generator that integrates the respiratory signal and generates the trigger signal when the integrated respiratory signal has a value representing a selected point in the subject's breathing cycle.

9. The medical data acquisition system, as set forth in claim 8, wherein the value representing a selected point in the subject's breathing cycle is selected to correspond to a point in the cycle where the motion of the lungs is at a minimum.

10. The medical data acquisition system, as set forth in claim 8, wherein the medical data system is an ultrasound system.

11. The medical data acquisition system, as set forth in claim 8, wherein the medical data system is a tomographic system.

12. The medical data acquisition system, as set forth in claim 8, wherein the medical data system is a MRI system.

13. The medical data acquisition system, as set forth in claim 8, wherein the respiratory signal device outputs a digital value and the trigger generator comprises a processor configured to integrate the respiratory signal and cause the output of the trigger signal.

14. A trigger generator for supplying a trigger signal to a medical device based on a respiratory signal representing the flow of gas into and out of the subject's lungs during the subject's breathing cycle, the trigger generator comprising:
an integrator that integrates the respiratory signal and generates a corresponding integrated respiratory signal;
a trigger level source that outputs a trigger level representing the selected point in the subject's breathing cycle; and
a trigger level detector that compares the integrated respiratory signal and the trigger level and generates the trigger signal when the integrated respiratory signal enters into a predetermined relationship with the trigger level.

15. A trigger generator for supplying a trigger signal to a medical device, the trigger generator comprising:
a respiratory signal device associated with the subject that generates a respiratory signal representing the flow of gas into and out of the subject's lungs during the subject's breathing cycle; and
a trigger generator that calculates a differential of the respiratory signal and generates a trigger signal when the differential has a value representing a selected point in the subject's breathing cycle.
